(19) United States
(12) Patent Application Publication
(10) Pub. No.: US 2025/0241516 A1
(43) Pub. Date: Jul. 31, 2025

(54) DISTAL TIPS AND ELEVATORS FOR MEDICAL DEVICES AND RELATED METHODS

(71) Applicants: Boston Scientific Scimed, Inc., Maple Grove, MN (US); Boston Scientific Medical Device Limited, Galway (IE)

(72) Inventors: Aditya DHANOTIYA, Indore (IN); Shrikant Vasant RAUT, Pune (IN); Pooja Bhuvanesh KULKARNI, Pune (IN); James WELDON, Newton, MA (US)

(73) Assignees: Boston Scientific Scimed, Inc., Maple Grove, MN (US); Boston Scientific Medical Device Limited, Galway (IE)

(21) Appl. No.: 19/034,901

(22) Filed: Jan. 23, 2025

Related U.S. Application Data
(60) Provisional application No. 63/624,999, filed on Jan. 25, 2024.

(51) Int. Cl. A61B 1/00

(52) U.S. Cl. CPC A61B 1/00098

(57) ABSTRACT

Distal tips and elevators for medical devices, and related methods are described. A medical device may include a shaft having a working channel and a distal tip coupled to the shaft. The distal tip may include a housing having a slot in communication with the working channel. The slot may include a bottom opening formed in a bottom wall of the housing. The medical device may further include an elevator positioned within the slot. In a fully lowered configuration of the elevator, at least a portion of the elevator may extend through the bottom opening.

### CROSS-REFERENCE TO RELATED APPLICATIONS

This application claims the benefit of priority to U.S. Provisional Application No. 63/624,999, filed on Jan. 25, 2024, which is incorporated by reference herein in its entirety.

### TECHNICAL FIELD

Various aspects of this disclosure relate generally to distal tips and elevators of medical devices, and related methods. In particular, aspects of this disclosure relate to distal tips and elevators of medical devices adapted for accessing a target site during an endoscopic procedure, among other aspects.

### BACKGROUND

During a medical procedure, such as Endoscopic Retrograde Cholangiopancreatography (ERCP), an operator may position an endoscope adjacent to a major duodenal papilla of a patient, and extend one or more medical instruments through a working channel of the endoscope. An elevator may be disposed at a distal end of the endoscope and may be actuatable in order to change an orientation of the one or more medical instruments to extend the one or more medical instruments into the major duodenal papilla.

### SUMMARY

Each of the aspects disclosed herein may include one or more aspects of the features described in connection with any of the other disclosed aspects.

According to some aspects of the present disclosure, a medical device may include a shaft having a working channel and a distal tip coupled to the shaft. The distal tip may include a housing. The housing may include a slot in communication with the working channel and the slot may include a bottom opening formed in a bottom wall of the housing. The medical device may further include an elevator positioned within the slot. In a fully lowered configuration of the elevator, at least a portion of the elevator may extend through the bottom opening.

According to some aspects, the slot may further include a distal opening on a distal wall of the housing and a top opening on a top wall of the housing. In some examples, the medical device may further include a protrusion extending from a back surface of the elevator. In some examples, in the fully lowered configuration, the protrusion may be received within the bottom opening. In some examples, the elevator may include a guide surface configured to contact a medical instrument, and the back surface of the elevator may be opposite to the guide surface. In some examples, the protrusion may be elongated in an axial direction along a distal portion of the back surface. In some examples, the protrusion may have a rounded square shape. In some examples, a lateral width of the protrusion may be less than a lateral width of the back surface. In some examples, in the fully lowered configuration, portions of the back surface adjacent to the protrusion may contact an inner surface of the bottom wall. In some examples, the portions of the back surface adjacent to the protrusion may be on a first lateral side of the back surface and a second lateral side of the back surface. In some examples, a portion of the back surface that is proximal of the protrusion may contact the inner surface of the bottom wall. In some examples, in the fully lowered configuration, a medical instrument extending along a guide surface of the elevator may extend distally from the distal tip at an angle of approximately 0.0 degrees to approximately 5.0 degrees. In some examples, a distal end of the elevator may include a distal surface. The distal surface may intersect a guide surface at a first distal edge and a central portion of the first distal edge may be distal to side portions of the first distal edge. In some examples, the elevator may include a guide surface configured to contact a medical instrument and the guide surface may have a “V” shape. In some examples, the bottom opening may be continuous with a distal opening of the slot and the distal opening may be formed in a distal wall of the distal tip.

According to some aspects of the present disclosure, a medical device may include a shaft having a working channel and a distal tip coupled to the shaft. The distal tip may include a housing having a slot in communication with the working channel. The medical device may further include an elevator positioned within the slot. The elevator may include a first side surface having a first top edge and a first bottom edge, a second side surface having a second top edge and a second bottom edge, and a distal surface having a distal top edge and a distal bottom edge. The first top edge may be parallel to the first bottom edge. The second top edge may be parallel to the second bottom edge. The distal bottom edge may be straight and may extend from the first bottom edge to the second bottom edge. In some examples, the elevator may include a guide surface to contact a medical instrument. The guide surface may have a “C” shape. The distal top edge may be a distal edge of the guide surface, such that the distal top edge may have a “C” shape. In some examples, the housing may include a distalmost surface that may extend substantially perpendicularly to a longitudinal axis of the distal tip.

According to some aspects of the present disclosure, an elevator for a medical device may include a proximal end, a distal end, and a guide surface configured to contact a medical instrument. The guide surface may extend from the proximal end to the distal end. A distal end of the guide surface may include a first side groove intersecting a central groove at a first ridge and a second side groove intersecting the central groove at a second ridge. In some examples, a proximal end of the guide surface may extend parallel to a longitudinal axis of the elevator and may branch into the first side groove and the second side groove.

### BRIEF DESCRIPTION OF THE DRAWINGS

The accompanying drawings, which are incorporated in and constitute a part of this application, illustrate exemplary aspects of this disclosure and together with the description, serve to explain the principles of the present disclosure.

FIGS. 1A and 1B illustrate an exemplary medical device, in accordance with some aspects of this disclosure.

FIGS. 2A-2E illustrate features of an exemplary distal tip and an exemplary elevator for use with the exemplary medical device of FIGS. 1A and 1B, in accordance with some aspects of this disclosure.

FIGS. 3A-3C illustrate features of another exemplary distal tip and exemplary elevator for use with the exemplary medical device of FIGS. 1A and 1B, in accordance with some aspects of this disclosure.

FIGS. 4A-4D illustrate features of another exemplary distal tip and exemplary elevator for use with the exemplary medical device of FIGS. 1A and 1B, in accordance with some aspects of this disclosure.

FIGS. 5A-5C illustrate features of a further exemplary distal tip and exemplary elevator for use with the exemplary medical device of FIGS. 1A and 1B, in accordance with some aspects of this disclosure.

FIGS. 6A-6C illustrate features of yet another exemplary elevator for use with the exemplary medical device of FIGS. 1A and 1B, in accordance with some aspects of this disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although duodenoscopes are referenced herein for illustration purposes, it will be appreciated that the disclosure encompasses any suitable medical device configured to allow an operator to access and view internal body anatomy of a subject (e.g., patient) and/or to deliver medical instruments or accessory devices, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, endoscopes, bronchoscopes, gastroscopes, endoscopic ultrasonography (“EUS”) scopes, colonoscopes, ureteroscopes, laparoscopes, cystoscopes, aspiration scopes, sheaths, catheters, or any other suitable delivery device or medical device may be used in connection with the features described herein.

Features of the medical devices herein may improve accessibility to various target sites within a patient's body, e.g., a major duodenal papilla, among other aspects. According to some aspects of the present disclosure, the medical device may include a distal tip having an elevator to, e.g., change an orientation of one or more medical instruments. Features of the distal tip and/or of the elevator may facilitate angling or orienting one or more medical instruments towards a desired location, e.g., an angle formed between the elevator and a longitudinal axis of the medical device may range from approximately 0.0 degrees to 90.0 degrees. For example, features of the distal tip and/or of the elevator may facilitate extending one or more medical instruments along a substantially linear path and/or in a substantially straight configuration. According to some aspects of the present disclosure, the elevator may include one or more grooves to facilitate locking one or more medical instruments in place during a medical procedure. According to some aspects of the present disclosure, the elevator may include curved and/or smooth surfaces that may allow for one or more medical instruments to move and/or glide more easily along surfaces of the elevator.

Reference will now be made in detail to examples of the present disclosure described above and illustrated in the accompanying drawings. Wherever possible, the same reference numbers will be used throughout the drawings to refer to the same or like parts. Unless otherwise specified herein, features of any of the distal tips and elevators disclosed herein may be combined in any suitable fashion.

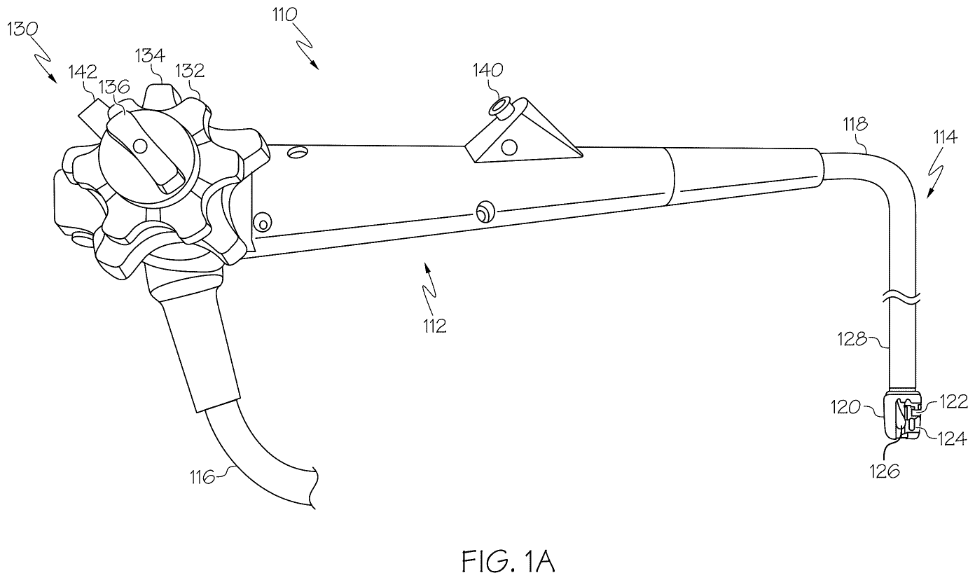

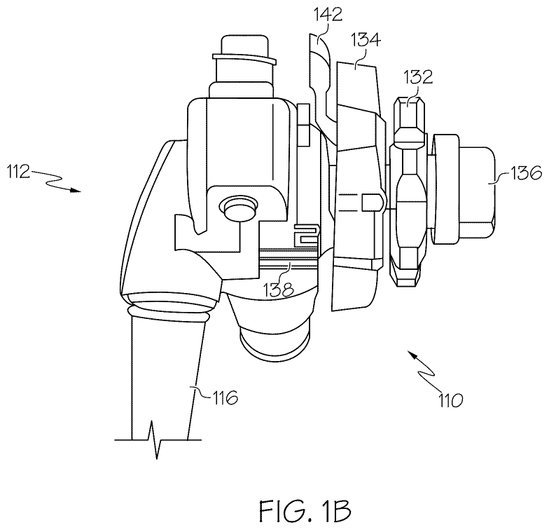

FIG. 1A depicts an exemplary medical device 110 (e.g., a duodenoscope) having a handle 112 and an insertion portion 114. FIG. 1B shows a proximal end of handle 112. Medical device 110 may also include an umbilicus 116 for purposes of connecting medical device 110 to sources of, e.g., air, water, suction, power, etc., as well as to image processing and/or viewing equipment. Insertion portion 114 may include a sheath or shaft 118 and a distal tip 120. Distal tip 120 may include an imaging device 122 (e.g., a camera) and a lighting source 124 (e.g., an LED or an optical fiber). In some examples, as shown in FIG. 1A, distal tip 120 may be side-facing (i.e., side viewing). That is, imaging device 122 and lighting source 124 may face radially outward, perpendicularly, approximately perpendicularly, or otherwise transverse to a longitudinal axis of shaft 118 and distal tip 120.

Distal tip 120 may also include an elevator 126 for changing an orientation of a medical instrument inserted in a working channel of medical device 110. Elevator 126 may alternatively be referred to as a swing stand, pivot stand, raising base, or any suitable other term. Elevator 126 may be rotatable via, e.g., an actuation wire or another control element that extends from handle 112, through shaft 118, to elevator 126.

A distal portion of shaft 118 that is connected to distal tip 120 may have a steerable section 128. Steerable section 128 may be, e.g., an articulation joint. Shaft 118 and steerable section 128 may include a variety of structures which are known or may become known in the art.

Handle 112 may have one or more actuators/control mechanisms 130. One or more of control mechanisms 130 may provide control over steerable section 128. One or more of control mechanisms may allow for provision of air, water, suction, etc. For example, handle 112 may include control knobs 132, 134 for left, right, up, and/or down control of steerable section 128. For example, one of knobs 132, 134 may provide left/right control of steerable section 128, and the other of knobs 132, 134 may provide up/down control of steerable section 128. Handle 112 may further include a first locking mechanism 136 and a second locking mechanism 142 (e.g., knobs or levers) for preventing steering and/or braking of steerable section 28 in at least one of an up, down, left, or right direction. Handle 112 may include a control lever 138 (see FIG. 1B). Control lever 138 may raise and/or lower elevator 126, via connection between control lever 138 and an actuating wire that extends from control lever 138, through shaft 118, to elevator 126. A port 140 may allow passage of a medical instrument through port 140, into a working channel of the medical device 110, through shaft 118, to distal tip 120.

Although FIG. 1A depicts a side-viewing medical device 110 (a device having imaging device 122 and/or lighting source 124 facing substantially radially outward), it will be appreciated that medical device 110 may alternatively be a forward-viewing device (a device having imaging device 122 and/or lighting source 124 facing substantially distally). In aspects, medical device 110 may include both a side-viewing imaging device 122 and/or lighting source 124 and a forward-facing imaging device 122 and/or lighting source 124.

In use, an operator may insert at least a portion of shaft 118 into a body lumen of a subject. Distal tip 120 may be navigated to a procedure site in the body lumen. The operator may insert a medical instrument (not shown) into port 140, and pass the medical instrument through shaft 118 via a working channel to distal tip 120. The medical instrument may exit the working channel at distal tip 120. The user may use control lever 138 to raise elevator 126 and angle the medical instrument toward a desired location (e.g., a papilla of the pancreas and biliary tract) to facilitate a medical procedure.

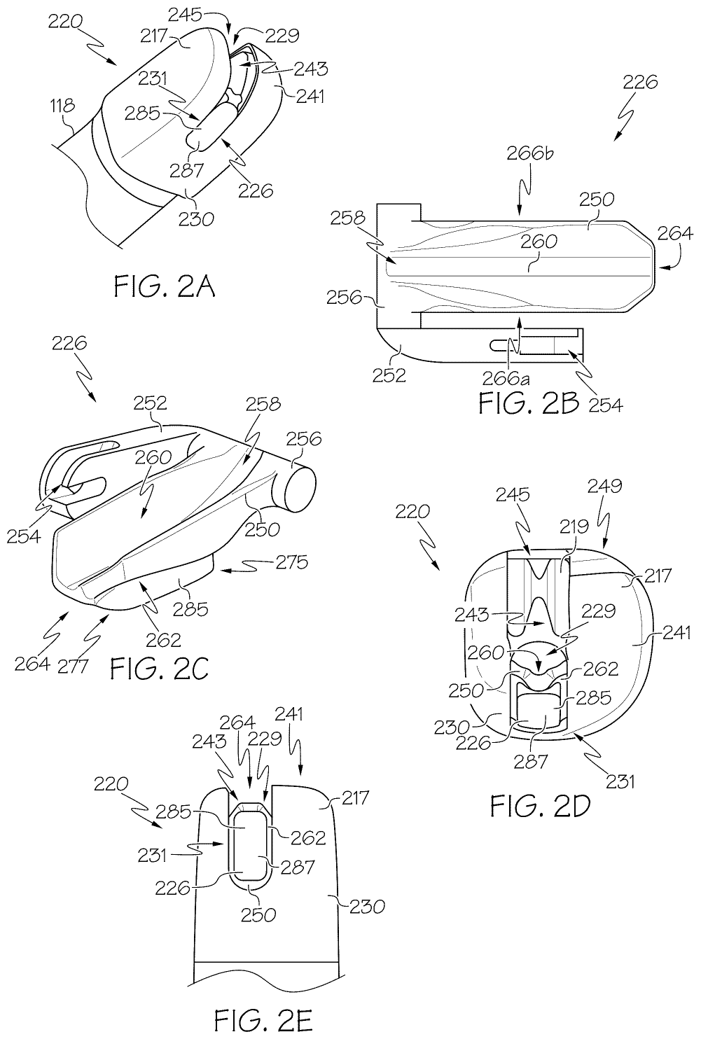

FIGS. 2A-2E illustrate an exemplary distal tip 220 that may include any of the features of distal tip 120, unless otherwise specified herein, and/or illustrate features of an exemplary elevator 226 of distal tip 220 that may include any of the features of elevator 126, unless otherwise specified herein. Distal tip 220 and elevator 226 may also have any of the features of any of the elevators disclosed below. Distal tip 220 may be used in combination with shaft 118 of medical device 110 (e.g., in place of distal tip 120).

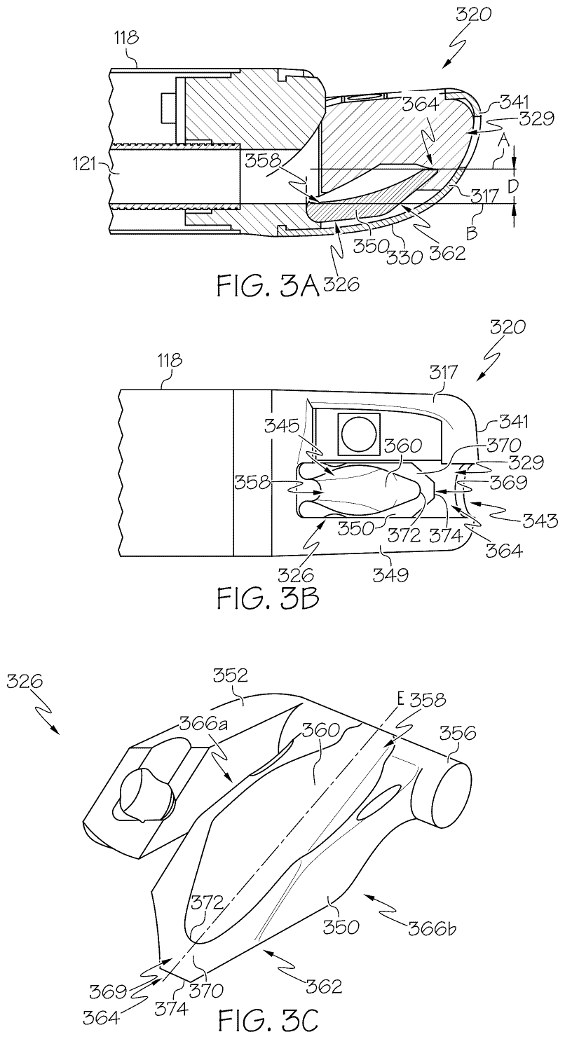

A housing 217 of distal tip 220 may include a slot 229, which may receive elevator 226, as shown in FIGS. 2A, 2D, and 2E. Slot 229 may extend approximately parallel to a central longitudinal axis of shaft 118/distal tip 220. As shown in FIG. 2D, slot 229 may be in communication with a working channel 121 (see FIGS. 3A, 4A, and 5A) of shaft 118. Slot 229 may be open on three sides (e.g., a top side adjacent to imaging device 122 (FIG. 1A)), a bottom side opposite of the top side, and a distal side), in addition to being in communication with a distal opening of working channel 121 (see, e.g., FIGS. 3A, 4A, and 5A). For example, slot 229 may include a bottom opening 231 formed on a bottom wall 230 of housing 217, a distal opening 243 formed on a distal wall 241 of housing 217, and a top opening 245 formed on a top wall 249 of housing 217. The terms “top,” “up,” “upward,” and the like refer to an upward direction of FIG. 2D (a direction in which imaging device 122 and/or lighting source 124 may generally face). The terms “bottom,” “down,” “downward,” and the like refer to a downward direction of FIG. 2D (opposite to the top/up/upward direction). Bottom opening 231 may be continuous with distal opening 243 and distal opening 243 may be continuous with top opening 245. In other words, a distalmost end of bottom opening 231 may be distal opening 243, and a distalmost end of top opening 245 may be distal opening 243. Slot 229 may extend through an entire thickness of distal tip 220, from top wall 249 to bottom wall 230. Distal opening 243 and top opening 245 of slot 229 may allow an operator to extend a medical instrument toward a target site at an angle ranging from approximately 0.0 degrees to approximately 90.0 degrees or more.

Elevator 226 may include a body 250 and an arm 252. Arm 252 may include a coupler 254 (e.g., an opening, slot, and/or other structure, shown in FIGS. 2B and 2C) for coupling a control wire to arm 252. Axle 256 (FIGS. 2B and 2C) may be at a proximal end of elevator 226 (e.g., a proximalmost end of elevator 226), coupled to a proximal end 258 of body 250. Proximal end 258 of body 250 may be opposite a distal end 264 of body 250. As shown in FIGS. 2A and 2E, distal end 264 of body 250 may be proximal of distal wall 241 of housing 217.

As the control wire is moved or urged distally or proximally, elevator 226 may rotate about an axle 256 (shown in FIGS. 2B and 2C). For example, moving the control wire proximally raises elevator 226 away from bottom opening 231 of slot 229 to a raised configuration. Moving the control wire distally lowers elevator 226 towards bottom opening 231 of slot 229 to a lowered configuration (e.g., to a fully lowered configuration as shown in FIGS. 2A, 2D, and 2E, and described in further detail below).

Body 250 may include a guide surface 260, which may be configured to contact and change an orientation of a medical instrument. Guide surface 260 may be on a front side of body 250 and may have an approximately “V” shape. As shown in FIG. 2D, distal tip 220 may include a locking surface 219 having a complementary shape to guide surface 260, such that guide surface 260 may mate with locking surface 219 (FIG. 2D) to lock a medical instrument in a desired position/configuration. Guide surface 260 may extend between proximal end 258 and distal end 264 of body 250, and between a first side 266a (closer to arm 252) and a second side 266b (farther from arm 252) of body 250 (see FIG. 2B). Body 250 may further include a back surface 262 (see, e.g., FIGS. 2D and 2E). Back surface 262 may be on an opposite side of body 250 from guide surface 260. When elevator 226 is in the fully lowered configuration, guide surface 260 may face upward/radially outward, in a generally same direction as that faced by imaging device 122 and lighting source 124 and toward top opening 245.

Elevator 226 may include a protrusion 285 extending from back surface 262. Protrusion 285 may be sized and shaped to be received within bottom opening 231 of slot 229. In other words, a size and a shape of protrusion 285 may be complementary to a size and a shape of bottom opening 231, such that protrusion 285 may fit within/mate with bottom opening 231. Protrusion 285 may extend downward in a direction perpendicular to a longitudinal axis of body 250, and may be elongated in an axial direction (e.g., a proximal/distal direction) along a portion, e.g., a distal portion, of back surface 262. Protrusion 285 may have a rounded rectangular, square, or ovular profile when viewed from below.

As shown particularly in FIG. 2C, a proximal end 275 of protrusion 285 may be distal of proximal end 258 of body 250, and a distal end 277 of protrusion 285 may be proximal of distal end 264 of body 250. As used herein, the term “lateral” may refer to a direction that is perpendicular to a longitudinal axis of distal tip 220, extending between first side 266a and second side 266b. A lateral width (a direction from first side 266a to second side 266b of body 250 perpendicular to a longitudinal axis of body 250 and perpendicular to a height of body 250 in an up/down direction) of protrusion 285 may be less than a lateral width of body 250. In other words, portions of back surface 262 that are recessed relative to and/or adjacent to protrusion 285 may be on either side (toward sides 266a, 266b) of protrusion 285. In alternatives, protrusion 285 may be omitted, and at least a portion of back surface 262 may extend through bottom opening 231.

As shown in FIGS. 2A, 2D, and 2E, when elevator 226 is in the fully lowered configuration, protrusion 285 is received within bottom opening 231 and portions of back surface 262 surrounding and/or adjacent to protrusion 285 (e.g., portions of back surface 262 proximal to protrusion 285 and/or portions of back surface 262 on either side of protrusion 285) may rest on or contact an inner surface of bottom wall 230 of housing 217. In other words, when elevator 226 is in the fully lowered configuration, side portions of back surface 262 may be to either side of bottom opening 231, and proximal portions of back surface 262 may be proximal of bottom opening 231. In the fully lowered configuration, a bottom surface 287 of protrusion 285 facing the same direction as back surface 262 may be aligned with an outer surface of bottom wall 230 of housing 217. Furthermore, in the fully lowered configuration, guide surface 260 may extend approximately horizontally/parallel to a central longitudinal axis of distal tip 220, such that a medical instrument may extended linearly in a distal direction (i.e., a straight configuration) along guide surface 260. In other words, a line extending longitudinally from distal end 264 of body 250 may form an angle of approximately 0.0 degrees with a line extending longitudinally from the bottom of working channel 121.

During an exemplary medical procedure, in the fully lowered configuration of elevator 226, an angle formed between a medical instrument extending along guide surface 260 (e.g., a guidewire) and a longitudinal axis of shaft 118/distal tip 220/working channel 121 may range from approximately 0.0 degrees to approximately 5.0 degrees, whereas a corresponding angle for a conventional elevator may be approximately 10.0 degrees. In the fully lowered configuration of elevator 226, a vertical distance (i.e., an upward direction perpendicular to the longitudinal axis of shaft 118/distal tip 220) from the medical instrument to an outermost face of imaging device 122 may be approximately 8.0 mm, whereas a corresponding distance for a conventional elevator may be approximately 4.0 mm. In a fully raised configuration of elevator 226, an angle formed between the medical instrument and the longitudinal axis of shaft 118/distal tip 220/working channel 121 may range from approximately 80.0 degrees to approximately 90.0 degrees.

FIGS. 3A-3C illustrate features of another exemplary distal tip 320 that may include any of the features of distal tips 120, 220, unless otherwise specified herein, and/or illustrate features of another exemplary elevator 326 that may include any of the features of elevators 126, 226, unless otherwise specified herein. Distal tip 320 and elevator 326 may also have any of the features of any of the elevators disclosed below. Distal tip 320 may be used in combination with shaft 118 of medical device 110 (e.g., in place of distal tip 120).

Elevator 326 may be received within a slot 329 (shown in FIGS. 3A and 3B) of a housing 317 of distal tip 320. Slot 329 may extend approximately parallel to a central longitudinal axis of shaft 118/distal tip 320. As shown in FIG. 3A, slot 329 may be in communication with working channel 121 of shaft 118. Slot 329 may be open on two sides (e.g., a top side adjacent to imaging device 122 and a distal side), in addition to being in communication with the distal opening of working channel 121. For example, slot 329 may include a distal opening 343 (FIG. 3B) formed on a distal wall 341 of housing 317 and a top opening 345 formed on a top wall 349 of housing 317. The terms “top,” “up,” “upward,” and the like refer to an upward direction of FIG. 3A. The terms “bottom,” “down,” “downward,” and the like refer to a downward direction of FIG. 3A.

Similar to elevator 226, elevator 326 may include an arm 352 and an axle 356 (FIG. 3C), having any of the features of arm 252 and axle 256, respectively. Elevator 326 may also include a body 350 including a proximal end 358 and a distal end 364. A guide surface 360 may be on a front side of body 350, and may extend between proximal end 358 and distal end 364 of body 350, and between first and second sides 366a, 366b (FIG. 3C) of body 350. Guide surface 360 may have a “V” shape, which may be deeper and/or more steeply angled than “V” shapes of conventional elevators (and/or guide surface 260). Body 350 may further include a back surface 362 on an opposite side of body 350 from guide surface 360.

Body 350 may have a shorter length (i.e., in a direction parallel to a longitudinal axis of body 350) than bodies of conventional elevators, which may facilitate extending a medical instrument along a guide surface 360 of elevator 326 in a substantially straight configuration. As shown in FIGS. 3A and 3B, body 350 may terminate proximal of distal opening 343 of slot 329. The shorter length of body 350 may allow for distal end 364 of body 350 to be relatively lower (i.e., closer to a bottom wall of a distal tip, e.g., a bottom wall 330 of distal tip 320) than a distal end of a conventional elevator in a fully lowered configuration. In other words, elevator 326 may lie flatter within distal tip 320 (or a conventional distal tip) than a conventional elevator in the fully lowered configuration. As shown in FIG. 3A, a distal end of housing 317 may curve upwards, e.g., bottom wall 330 may curve upwards to distal wall 341. The curvature of the distal end of housing 317 may necessitate a conventional elevator to have a higher distal end to fit within housing 317 (or a conventional distal tip).

FIG. 3A shows a first line A extending from a distal end of guide surface 360 (i.e., at distal end 364 of body 350). FIG. 3A also shows a second line B extending from the bottom of working channel 121, parallel to the central longitudinal axis of shaft 118/working channel 121. A distance D between first line C and second line B may be less than a distance between equivalent structures for conventional elevators. For example, a distance D may be approximately 2.7 mm, whereas a corresponding distance for a conventional elevator may be approximately 4.0 mm. In other words, a distalmost end of guide surface 360 of elevator 326 may be lower or closer to bottom wall 330 of distal tip 320 than a longer, conventional elevator would be.

As shown in FIG. 3C, distal end 364 of body 350 may extend between first side 366a of body 350 and second side 366b of body 350. Distal end 364 may extend further in a distal direction at a central portion 369 of body 350 (at or near a central longitudinal axis E of body 350) than at first side 366a and second side 366b. Distal end 364 may include a substantially planar distal surface 370 that extends between guide surface 360 and back surface 362. Distal surface 370 may have a substantially U or rounded V shape.

Distal surface 370 may intersect guide surface 360 at a first distal edge 372. A central portion of edge 372 (near central portion 369 of body 350) may be distal to side portions of first distal edge 372 near sides 366a, 366b of body 350. Distal surface 370 may intersect back surface 362 at a second distal edge 374. A central portion of second distal edge 374 (near central portion 369 of body 350) may be distal to side portions of second distal edge 374 near sides 366a, 366b of body 350. All portions of distal edge 372 may be proximal to respective portions of second distal edge 374. For example, a distalmost portion of second distal edge 374 may be distal to a distalmost portion of first distal edge 372. Thus, distal surface 370 may be angled toward back surface 362 extending in a distal direction.

During an exemplary medical procedure, in the fully lowered configuration of elevator 326, an angle formed between a medical instrument (e.g., a guidewire) extending along guide surface 360 and a longitudinal axis of shaft 118/distal tip 320/working channel 121 may range from approximately 0.0 degrees to approximately 5.0 degrees. In the fully lowered configuration of elevator 326, a vertical distance (described above) from the medical instrument having a smaller radius to the outermost face of imaging device 122 may be approximately 6.0 mm. In a fully raised configuration of elevator 326, an angle formed between the medical instrument having a smaller radius (e.g., a guidewire) and the longitudinal axis of shaft 118/distal tip 320/working channel 121 may be approximately 70.0 degrees. In the fully raised configuration of elevator 326, an angle formed between a medical instrument having a larger radius (e.g., a tome) and the longitudinal axis of shaft 118/distal tip 320 may range from approximately 60.0 degrees to approximately 70.0 degrees.

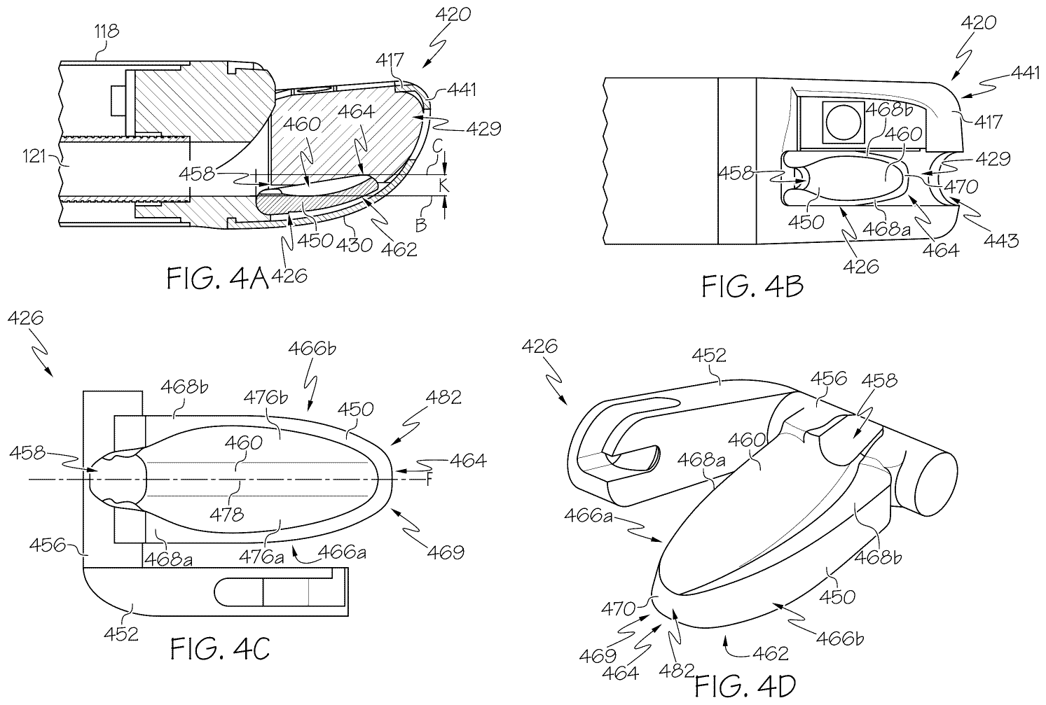

FIGS. 4A-4D illustrate features of another exemplary distal tip 420 that may include any of the features of distal tips 120, 220, 320, unless otherwise specified herein, and/or illustrate features of another exemplary elevator 426 that may include any of the features of elevators 126, 226, 326, unless otherwise specified herein. Distal tip 420 and elevator 426 may also have any of the features of any of the elevators disclosed below. Distal tip 420 may be used in combination with shaft 118 of medical device 110 (e.g., in place of distal tip 120).

Elevator 426 may be received within a slot 429 (shown in FIGS. 4A and 4B) of a housing 417 of distal tip 420. Slot 429 may include any of the features of slot 329. Similar to elevators 226, 326, elevator 426 may include an arm 452 and an axle 456, having any of the features of arms 252, 352 and axles 256, 356, respectively. Elevator 426 may also include a body 450 including a proximal end 458 and a distal end 464. A guide surface 460 may be on a front side of body 450, and may extend between proximal end 458 and distal end 464 of body 450, and between first and second sides 466a, 466b (FIGS. 4C and 4D) of body 450. Body 450 may further include a back surface 462 on an opposite side of body 450 from guide surface 460. Similar to body 350, body 450 may have a shorter length (i.e., in a direction parallel to a longitudinal axis of body 450) than bodies of conventional elevators, and may terminate proximal of a distal opening 443 of slot 429.

Body 450 may extend along bottom wall 430 (FIG. 4A) of housing 417 and may not extend along distal wall 441 of housing 417. In other words, a curvature of body 450 from proximal end 458 to distal end 464 may be complementary to a curvature of a bottom wall 430 of housing 417, which may further facilitate extending a medical instrument along guide surface 460 of elevator 426 in a substantially straight configuration. For example, in the fully lowered configuration, an entirety of back surface 462 of body 450 may be in close proximity to (e.g., touching or almost touching) an inner surface of bottom wall 430. FIG. 4A shows second line B (described above) and a third line C extending from a distal end of guide surface 460 (i.e., at distal end 464 of body 450). A distance K between second line B and third line C may be less than a distance D between first line A and second line B for elevator 326. In other words, a distalmost end of guide surface 460 of elevator 426 may be closer to bottom wall 430 of distal tip 420 than elevator 326 may be to bottom wall 330 of distal tip 320. For example, distance K may be approximately 1.5 mm.

As shown particularly in FIGS. 4B and 4C, a proximal portion of body 450 may be wider than a distal portion of body 450. In other words, distal end 464 of body 450 may taper to a central distal portion 482. Central distal portion 482 may be at a central portion 469 of body 450 (at or near a central longitudinal axis F of body 450), Body 450 may include a first side edge 468a (closer to first side 466a) and a second side edge 468b (closer to second side 466b). Proximal portions of first and second side edges 468a, 468b may extend substantially parallel to central longitudinal axis F, and distal portions of first and second side edges 468a, 468b may curve inward towards central longitudinal axis F/central distal portion 482. A distal surface 470 at a distalmost end of elevator 426 may extend between guide surface 460 and back surface 462. Distal surface 470 may curve outward from guide surface 460 to back surface 462.

In accordance with tapered distal end 464 of body 450, guide surface 460 may have a tapered distal end, such that a central portion 478 of guide surface 460 at central portion 469 of body 450 extends farther distally than side portions 476a (closer to first side 466a), 476b (closer to second side 466b) of guide surface 460. Central portion 478 of guide surface 460 may be recessed along a height (i.e., in a direction upward and perpendicular to a longitudinal axis of body 450) of elevator 426 relative to side portions 476a, 476b of guide surface 460, such that guide surface 460 may be concave. Surfaces and edges of body 450 (e.g., guide surface 460, distal surface 470, first side edge 468a, second side edge 468b, etc.) may be smooth and/or atraumatic to allow medical instruments of varying sizes to glide more easily along surfaces of elevator 426 (i.e., with less resistance) during a medical procedure.

During an exemplary medical procedure, in a fully lowered configuration of elevator 426, an angle formed between a medical instrument having a smaller radius (e.g., a guidewire) extending along guide surface 460 and a longitudinal axis of shaft 118/distal tip 420/working channel 121 may range from approximately 0.0 degrees to approximately 5.0 degrees. In the fully lowered configuration of elevator 426, a vertical distance (described above) from the medical instrument having a smaller radius to the outermost face of imaging device 122 may be approximately 6.0 mm. In a fully raised configuration of elevator 426, an angle formed between the medical instrument having a smaller radius (e.g., a guidewire) and the longitudinal axis of shaft 118/distal tip 420 may be approximately 60.0 degrees. In the fully raised configuration of elevator 426, an angle formed between a medical instrument having a larger radius (e.g., a tome) and the longitudinal axis of shaft 118/distal tip 420/working channel 121 may be approximately 70.0 degrees.

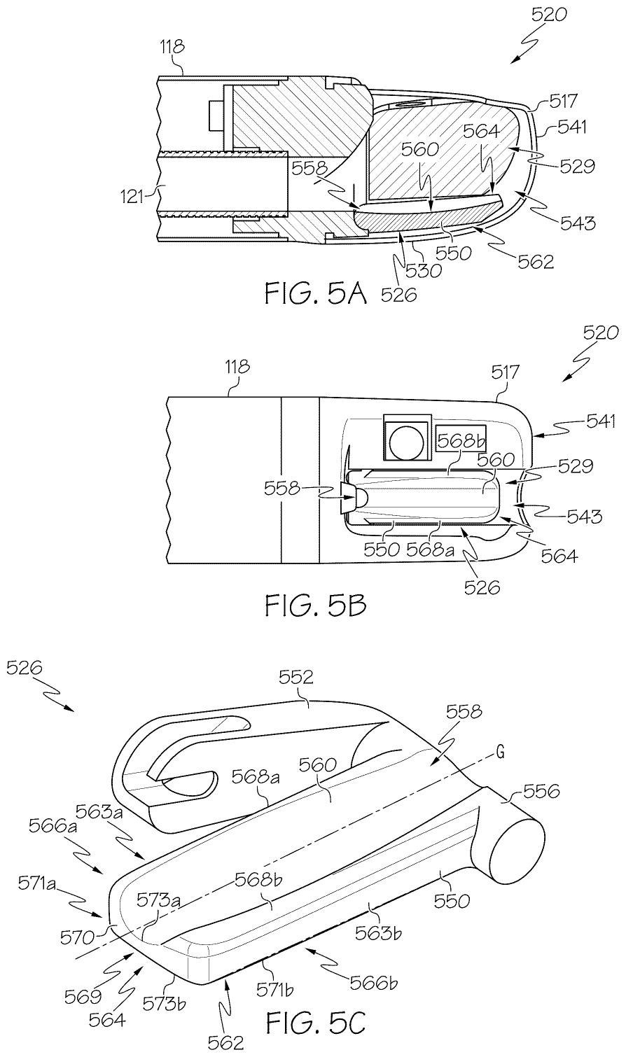

FIGS. 5A-5C illustrate features of another exemplary distal tip 520 that may include any of the features of distal tips 120, 220, 320, 420, unless otherwise specified herein, and/or illustrate features of another exemplary elevator 526 that may include any of the features of elevators 126, 226, 326, 426, unless otherwise specified herein. Distal tip 520 and elevator 526 may also have any of the features of any of the elevators disclosed below. Distal tip 520 may be used in combination with shaft 118 of medical device 110 (e.g., in place of distal tip 120).

Elevator 526 may be received within a slot 529 (shown in FIGS. 5A and 4B) of a housing 517 of distal tip 520. Slot 529 may include any of the features of slots 329, 429. Similar to elevators 226, 326, 426, elevator 526 may include an arm 552 and an axle 556 (FIG. 5C), having any of the features of arms 252, 352, 452 and axles 256, 356, 456, respectively. Elevator 526 may also include a body 550 including a proximal end 558 and a distal end 564. A guide surface 560 may be on a front side of body 550, and may extend between proximal end 558 and distal end 564 of body 550, and between first and second sides 566a, 566b of body 550. Body 550 may further include a back surface 562 on an opposite side of body 550 from guide surface 560. As shown in FIGS. 5A and 5B, body 550 may terminate proximal of a distal opening 543 of slot 529.

Guide surface 560 may cover a greater surface area of elevator 526 compared to guide surfaces 260, 360, 460 of elevators 226, 326, 426. Therefore, guide surface 560 may accommodate larger medical instruments. In other words, guide surface 560 may be wider than guide surfaces 260, 360, 460. Guide surface 560 may have a substantially C or U shape. Back surface 562 may be substantially planar. Body 550 may have a greater length (i.e., in a direction parallel to a longitudinal axis of body 550) than bodies of conventional elevators and/or elevators 226, 326, or 426. The greater length of body 550 may provide greater mechanical advantage in raising elevator 526 using lever 138. Elevator 526 and distal tip 520 may provide three points of contact for an instrument when elevator 526 is raised/locked: a distal surface at a top of working channel 121, distal end 564 of elevator 526, and a point along an axially middle portion of guide surface 560.

A thickness of body 550 (i.e., in an up/down direction perpendicular to a longitudinal axis of body 550) may be less than bodies of conventional elevators, which may facilitate extending a medical instrument in a substantially straight configuration. A bottom wall 530 (FIG. 5A) of housing 517 may extend substantially parallel to a central longitudinal axis of shaft 118/distal tip 520/working channel 121. As shown in FIG. 5A, in a fully lowered configuration, back surface 562 of body 550 may contact or rest on an inner surface of bottom wall 530, such that a line extending from a bottom of working channel 121 (e.g., line B, shown in FIGS. 3A and 4A) may be substantially aligned/coaxial with a line extending distally from guide surface 560. In other words, a distance between a bottom surface of working channel 121 and a distal end of guide surface 560 may be approximately 0.0 mm.

Body 550 may include a first side surface 563a (closer to first side 566a) and a second side surface 563b (closer to second side 566b). First and second side surfaces 563a, 563b may extend substantially parallel to a central longitudinal axis G of body 550 from proximal end 558 to distal end 564 of body 550. First side surface 563a may extend from a first top edge 568a (i.e., on a same top side as guide surface 560) to a first bottom edge 571a (i.e., on a same bottom side as back surface 562; see FIG. 5A). Second side surface 563b may extend from a second top edge 568b to a second bottom edge 571b. First top edge 568a may be parallel to first bottom edge 571a and second top edge 568b may be parallel to second bottom edge 571b. A lateral width of body 550 (i.e., a direction from first side 566a to second side 566b of body 550 perpendicular to a longitudinal axis of body 550 and perpendicular to a height of body 250 in the up/down direction) may be substantially uniform from proximal end 558 to distal end 564 of body 550.

Distal end 564 may extend from first side 566a to second side 566b, and may include a substantially planar distal surface 570 that extends between guide surface 560 and back surface 562. For example, guide surface 560 and distal surface 570 may intersect at a distal top edge 573a, and back surface 562 and distal surface 570 may intersect at a distal bottom edge 573b. Distal bottom edge 573b may be straight and may extend from first bottom edge 571a to second bottom edge 571b. Distal top edge 573a may be a distal edge of guide surface 560 and may have a C or U shape in accordance with the C or U shape of guide surface 560. In accordance with the C or U shape of guide surface 560, and the planar nature of back surface 562, side portions of distal surface 570 near sides 566a, 566b of body 550 may be thicker (i.e., in a direction perpendicular to a longitudinal axis of body 550) than a central portion of distal surface 570 at a central portion 569 of body 550 (at or near central longitudinal axis G).

A distal wall 541 of housing 517 may extend substantially upward and perpendicular to the central longitudinal axis of shaft 118/distal tip 520/working channel 121. For example, distal wall 541 may be near to and distal to distal end 564 of body 550. Whereas distal walls 241, 341, 441 may extend distally and upwardly, distal wall 541 may extend substantially upwardly, without extending substantially in a distal direction. In some examples, an upper portion of distal wall 541 may curve slightly in a proximal direction. A shape of distal wall 541 may mean that an axial/longitudinal length of distal tip 520 is less than an axial/longitudinal length of distal tips 220, 320, 420. A shape of distal wall 541 may provide a softer, more blended surface as compared to distal walls 241, 341, 441, which may provide for smoother gliding and may to help prevent tissue damage during advancement of distal tip 520 through an anatomy.

During an exemplary medical procedure, in the fully lowered configuration of elevator 526, an angle formed between a medical instrument (e.g., a guidewire) extending along guide surface 560 and a longitudinal axis of shaft 118/distal tip 520/working channel 121 may range from approximately 0.0 degrees to approximately 5.0 degrees. In the fully lowered configuration of elevator 526, a vertical distance (described above) from the medical instrument to the outermost face of imaging device 122 may be approximately 8.0 mm. In a fully raised configuration of elevator 526, an angle formed between the medical instrument and the longitudinal axis of shaft 118/distal tip 520/working channel 121 may be approximately 90.0 degrees.

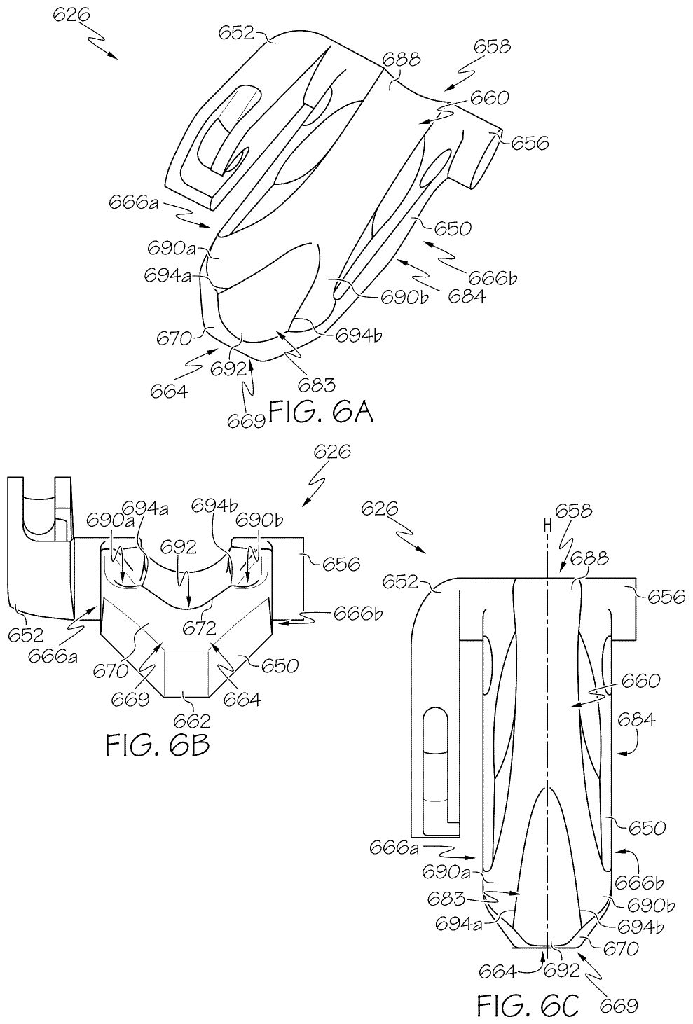

FIGS. 6A-6C illustrate features of another exemplary elevator 626 that may include any of the features of elevators 126, 226, 326, 426, 526, unless otherwise specified herein. Elevator 626 may be used in place of elevators 126, 226, 326, 426, 526 in distal tips 120, 220, 320, 420, 520. Similar to elevators 226, 326, 426, 526, elevator 626 may include an arm 652 and an axle 656, having any of the features of arms 252, 352, 452, 552 and axles 256, 356, 456, 556, respectively. Elevator 626 may also include a body 650 including a proximal end 658 and a distal end 664. A guide surface 660 may be on a front side of body 650, and may extend between proximal end 658 and distal end 664 of body 650, and between first and second sides 666a, 666b of body 650. Body 650 may further include a back surface 662 (FIG. 6B) on an opposite side of body 650 from guide surface 660.

A main portion 688 of guide surface 660 may extend from proximal end 658 to an intermediate portion of body 650 between proximal end 658 and distal end 664 of body 650. Main portion 688 may extend parallel to a central longitudinal axis H of body 650 and at a central portion 669 of body 650 (at or near central longitudinal axis H). Guide surface 660 may widen beyond or distally of an intermediate portion 684 of body 650. In other words, a distal portion of guide surface 660 may be wider than a proximal portion of guide surface 660.

Beyond intermediate portion 684, main portion 688 of guide surface 660 may diverge into or branch into a plurality of grooves. For example, a distal portion 683 of guide surface 660 may include a first side groove 690a (closer to first side 666a), a second side groove 690b (closer to second side 666b), and a central groove 692 between first and second side grooves 690a, 690b.

First side groove 690a and central groove 692 may intersect at a first ridge 694a. Second groove 690b and central groove 692 may intersect at a second ridge 694b. As shown in FIGS. 6A and 6C, first side groove 690a may curve from main portion 688 toward first side 666a, and second side groove 690b may curve from main portion 688 toward second side 666b. In other words, first side groove 690a may be angled toward first side 666a (away from central longitudinal axis H) and second side groove 690b may be angled toward second side 666b (away from central longitudinal axis H). Central groove 692 may extend between first and second ridges 694a, 694b, and may widen from a proximal end of central groove 692 (i.e., an end of central groove 692 adjacent to main portion 688) to a distal end (i.e., an end of central groove 692 adjacent to a distal surface 670 of body 650) of central groove 692. The distal end of central groove 692 may extend farther distally than first and second grooves 690a, 690b.

Distal end 664 of body 650 may extend from first side 666a to second side 666b, and may include a distal surface 670 that extends between guide surface 660 and back surface 662. Side portions of distal surface 670 near first and second sides 666a, 666b of body 650 may be proximal to a central portion of distal surface 670 at central portion 669 of body 650. Distal surface 670 may intersect guide surface 660 at a distal edge 672 (see, e.g., FIG. 6B). For example, first side groove 690a and distal surface 670 may intersect at the side portion of distal edge 672 near first side 666a. Second side groove 690b and distal surface 670 may intersect at the side portion of distal edge 672 near second side 666b. Central groove 692 and distal surface 670 may intersect at the central portion of distal edge 672 at central portion 669 of body 650. As particularly shown in FIG. 6B, the central portion of distal edge 672 extends further downward (i.e., in a direction towards back surface 662 and perpendicular to a longitudinal axis of body 650) than the side portions of distal edge 672. In other words, distal portions of central groove 692 may be deeper than side grooves 690a, 690b.

Side grooves 690a, 690b and central groove 692 may be used to angle or orient one or more medical instruments toward a target a site. Furthermore, side grooves 690a, 690b and central groove 692 may be used to lock one or more medical instruments in a desired angle/position. For example, during an exemplary medical procedure, an operator may extend a medical instrument along main portion 688, and may further extend the medical instrument along first side groove 690a to lock the medical instrument in place within first side groove 690a. The operator may then extend another medical instrument to lock the another medical instrument within central groove 692 or second side groove 690b. In examples, one of side grooves 690a, 690b (e.g., side groove 690a) may be used to angle a medical instrument toward a pancreatic duct, and the other of side grooves 690a, 690b may be used to angle a medical instrument toward a common bile duct (e.g., side groove 690b). Alternatively or additionally, a medical instrument may be extended along central groove 692 to lock the medical instrument in a neutral position (not extending off to one side 666a, 666b or the other side 666a, 666b). Thus, an operator may lock one or more medical instruments in one or more of side grooves 690a, 690b and/or central groove 692.

Aspects of the above distal tips and elevators may be combined in any combination or subcombination. For example, any of the distal tips disclosed herein may include a bottom opening similar to bottom opening 231, and any of the elevators disclosed herein may include a protrusion similar to protrusion 285. Similarly, any of the distal tips disclosed herein may have the shortened length of distal tip 520. As another example, any of the elevators disclosed herein may have any of the aspects of guide surface 660.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical device comprising:
a shaft comprising a working channel;
a distal tip coupled to the shaft and comprising a housing, wherein the housing includes a slot in communication with the working channel, wherein the slot includes a bottom opening formed in a bottom wall of the housing; and
an elevator positioned within the slot, wherein, in a fully lowered configuration of the elevator, at least a portion of the elevator extends through the bottom opening.

2. The medical device of claim 1, wherein the slot further includes a distal opening on a distal wall of the housing, and a top opening on a top wall of the housing.

3. The medical device of claim 1, further comprising a protrusion extending from a back surface of the elevator.

4. The medical device of claim 3, wherein, in the fully lowered configuration, the protrusion is received within the bottom opening.

5. The medical device of claim 3, wherein the elevator includes a guide surface configured to contact a medical instrument, and wherein the back surface is opposite to the guide surface.

6. The medical device of claim 3, wherein the protrusion is elongated in an axial direction along a distal portion of the back surface.

7. The medical device of claim 6, wherein the protrusion has a rounded square shape.

8. The medical device of claim 3, wherein a lateral width of the protrusion is less than a lateral width of the back surface.

9. The medical device of claim 8, wherein in the fully lowered configuration, portions of the back surface adjacent to the protrusion contact an inner surface of the bottom wall.

10. The medical device of claim 9, wherein the portions of the back surface adjacent to the protrusion are on a first lateral side of the back surface and a second lateral side of the back surface.

11. The medical device of claim 10, wherein a portion of the back surface that is proximal of the protrusion contacts the inner surface of the bottom wall.

12. The medical device of claim 1, wherein in the fully lowered configuration, a medical instrument extending along a guide surface of the elevator extends distally from the distal tip at an angle of approximately 0.0 degrees to approximately 5.0 degrees.

13. The medical device of claim 1, wherein a distal end of the elevator includes a distal surface, wherein the distal surface intersects a guide surface at a first distal edge, and wherein a central portion of the first distal edge is distal to side portions of the first distal edge.

14. The medical device of claim 1, wherein the elevator includes a guide surface configured to contact a medical instrument, and wherein the guide surface has a “V” shape.

15. The medical device of claim 1, wherein the bottom opening is continuous with a distal opening of the slot, wherein the distal opening is formed in a distal wall of the distal tip.

16. A medical device comprising:
a shaft comprising a working channel;
a distal tip coupled to the shaft and comprising a housing, wherein the housing includes a slot in communication with the working channel; and
an elevator positioned within the slot, wherein the elevator includes
a first side surface having a first top edge and a first bottom edge;
a second side surface having a second top edge and a second bottom edge; and
a distal surface having a distal top edge and a distal bottom edge;
wherein the first top edge is parallel to the first bottom edge, wherein the second top edge is parallel to the second bottom edge, and wherein the distal bottom edge is straight and extends from the first bottom edge to the second bottom edge.

17. The medical device of claim 16, wherein the elevator includes a guide surface to contact a medical instrument, wherein the guide surface has a “C” shape, and wherein the distal top edge is a distal edge of the guide surface, such that the distal top edge has a “C” shape.

18. The medical device of claim 16, wherein the housing includes a distalmost surface that extends substantially perpendicularly to a longitudinal axis of the distal tip.

19. An elevator for a medical device, comprising:
a proximal end; a distal end; and
a guide surface configured to contact a medical instrument and extending from the proximal end to the distal end; and wherein a distal end of the guide surface includes a first side groove intersecting a central groove at a first ridge and a second side groove intersecting the central groove at a second ridge.

20. The elevator of claim 19, wherein a proximal end of the guide surface extends parallel to a longitudinal axis of the elevator and branches into the first side groove and the second side groove.
